(19) United States
(12) Patent Application Publication
(10) Pub. No.: US 2004/0064008 A1
(43) Pub. Date: Apr. 1, 2004

(54) Molecular sieve catalyst composition

(76) Inventors: Torsten Maurer, Frankenthal (DE); Bettina Kraushaar-Czarnetzki, Karlsruhe (DE)

Correspondence Address: EXXONMOBIL CHEMICAL COMPANY, P O BOX 2149, BAYTOWN, TX, 77522-2149, US

(21) Appl. No.: 10/260,178

(22) Filed: Sep. 30, 2002

(51) Int. Cl. C07C 1/00; B01J 29/06

(52) U.S. Cl. 585/640; 502/64; 502/71; 502/77; 502/78; 502/79; 502/214

(57) ABSTRACT

A catalyst composition comprising molecular sieve particles in a matrix, at least 75% of the pore volume of the composition, as measured by mercury porosimetry, having a pore size of at most 20 nm, as well as processes for the manufacture of such catalysts and their use as catalysts, especially for methanol to hydrocarbon processes. These catalyst compositions have a high proportion of mesopores (pores of at most 20 nm as measured by mercury porosimetry).

### FIELD OF THE INVENTION

This invention relates to a molecular sieve catalyst composition, a method of making the composition, and to a process using the catalyst composition.

### BACKGROUND OF THE INVENTION

The conversion of oxygenates to hydrocarbons is an important process for the petrochemical industry. The oxygenates, of which methanol is a common example, may themselves be obtained from a variety of sources by a variety of reactions. Such a source, for example natural gas, petroleum liquids, coal, recycled plastics, may be converted to synthesis gas which in turn is converted to methanol, the preferred oxygenate for light olefin manufacture. Light olefins, especially ethylene and propylene, are important commodity petrochemicals for the manufacture of plastics, plasticizers, lubricants, and other commercially important materials.

Conversion of oxygenates to hydrocarbons, especially methanol to olefins, is usually carried out in the presence of a molecular sieve catalyst.

Molecular sieves, especially crystalline molecular sieves, both natural and synthetic, of many types, are known per se and as catalysts for conversion of one type of organic material to another or others. Molecular sieves may notionally be divided into two types, zeolitic, which are based on an aluminosilicate atomic framework, and non-zeolitic, which include the SAPO's and ALPO's. Examples of molecular sieve materials suitable for catalysing oxygenate to hydrocarbon conversions (hereinafter occasionally referred to for simplicity as methanol to hydrocarbon, especially to olefin, or for brevity as MTH or MTO, conversions) include more especially MFI, CHA, and AEI.

Molecular sieve catalysts are typically formed into compositions comprising the molecular sieve, binder, and/or a matrix material, which has various beneficial results. Some molecular sieves are more active as catalysts than may be required, and varying their proportions in a catalyst composition enables their activity to be controlled. A matrix, or a binder, may itself be a catalyst, active either in the reaction, e.g., the MTO conversion, that the molecular sieve is to catalyse, or in some other reaction that precedes or succeeds the MTO reaction, facilitating the overall reaction in some way. The binder or matrix also provides a mechanical function to limit the breakdown of the catalyst particles during the course of a reaction. For example, in a fluidized bed reactor, the collisions between catalyst composition particles themselves and with the reactor walls cause attritive breakdown of the composition, the resulting fines leaving the reactor and causing problems downstream. The binder or matrix may also control the access of reactants to, or the escape of reaction products from, the catalyst by the size of its pores, the size of the matrix particles, or otherwise.

Examination of the porosity of catalyst compositions using porosimetry reveals pores characterized as macropores, mesopores, and micropores. Different workers place different values on the pore size boundaries between mesopores and macropores. In this specification, micropores are those of diameter less than 2 nm (the limit of penetration when using mercury porosimety), mesopores are those of diameter between 2 and 20 nm, and macropores are those of diameter greater than 20 nm. The reader is referred to “Analytical Methods in Fine Particle Technology”, P. A. Webb, C. Orr, Micromeretics Instrument Corporation, 1997, ISBN 0-9656783-0-X, incorporated by reference, for details of mercury porosimetry.

It has been found that advantages result when a substantial proportion of the pore volume, as measured by mercury porosimetry, and hence pores of diameter of 2 nm and above, is in mesopores and the proportion of pore volume in macropores is limited. These advantages, which will be discussed in more detail below, include higher conversion at constant selectivity, catalyst life, and crush strength.

### SUMMARY OF THE INVENTION

The present invention accordingly provides a catalyst composition comprising molecular sieve catalyst particles in a matrix, at least 75% of the pore volume of the composition being of pore size at most 20 nm.

As indicated above, pore size is measured by mercury porosimetry, and references to measurements of pore sizes and percentages of pore volume represented by pores of a given size or in a given size range are to measurements carried out in that way. Accordingly, they do not take into account the micropores that are present in the molecular sieve particles themselves.

The present invention also provides a process for the manufacture of a catalyst composition according to the invention, which comprises mixing molecular sieve particles with a binder and optionally an additional matrix-forming material, under mixing conditions such that at least 75% of the pore volume, as measured by mercury porosimetry, of the composition is of pore size at most 20 nm.

The invention also provides a process for converting a feedstock, especially an oxygenate-containing feedstock, into a hydrocarbon, especially an olefin, -containing product in the presence of the catalyst composition of the invention.

The invention further provides the use of a catalyst composition according to the invention to enhance catalyst life or reduce deactivation rate in converting a feedstock, especially an oxygenate-containing feedstock, to a hydrocarbon, especially an olefin, -containing product. Reduction of deactivation rate is important in both fixed and fluidized bed reactions.

The invention still further provides the use of a catalyst composition according to the invention to enhance conversion rate in converting a feedstock, especially an oxygenate-containing feedstock, to a hydrocarbon, especially an olefin, -containing product.

### BRIEF DESCRIPTION OF THE DRAWING

The present invention will be better understood by reference to the Detailed Description of the Invention when taken together with the attached drawing, wherein

FIG. 1 shows the results of two methanol to olefins conversion experiments with periodical methanol flow interruptions of 10 minutes after 15 min time on stream.

### DETAILED DESCRIPTION OF THE INVENTION

The present invention accordingly provides a catalyst composition comprising molecular sieve catalyst particles in a matrix, at least 75% of the pore volume of the composition being of pore size at most 20 nm.

Advantageously, at least 80%, preferably at least 90%, and more preferably at least 95%, of the pore volume is of pore size at most 20 nm. Advantageously, the mean pore size is within the range of from 4 to 15 nm, preferably from 4 to 14 nm, and more preferably within the range of 6 to 12 nm.

As molecular sieve there may be mentioned, using the terminology of the Atlas of Zeolite Framework Types, 5^{th} Edition, Elsevier, 2001, the small pore molecular sieves of framework types AEI, AFT, APC, ATN, ATT, ATV, AWW, BIK, CAS, CHA, CHI, DAC, DDR, EDI, ERI, GOO, KFI, LEV, LOV, LTA, MON, PAU, PHI, RHO, ROG, THO, and substituted forms thereof; the medium pore molecular sieves of framework types AEL, AFO, EUO, FER, HEU, MEL, MFI, MTW, MTT, TON, and substituted forms thereof; and the large pore molecular sieves of framework types AFI, EMT, FAU, and substituted forms thereof. Other molecular sieves are of framework types ANA, BEA, CFI, CLO, DON, GIS, LTL, MER, MOR, MWW and SOD. Examples of the preferred molecular sieves, particularly for converting an oxygenate-containing feedstock into hydrocarbons, especially olefins, are of types AEL, AFI, BEA, CHA, ERI, FAU, FER, GIS, LTA, LTL, MER, MFI, MOR, MTT, MWW, TAM and TON.

A preferred aluminosilicate or zeolite molecular sieve is one of the MFI framework type, ZSM-5.

Among the aluminum and phosphorus-containing and aluminum, phosphorus, and silicon-containing molecular sieves there may be mentioned aluminophosphate (ALPO) molecular sieves and silicoaluminophosphate (SAPO) molecular sieves and substituted, preferably metal substituted, ALPO and SAPO molecular sieves. The most preferred molecular sieves are SAPO molecular sieves, and metal substituted SAPO molecular sieves.

Examples of SAPO and ALPO molecular sieves are one or a combination of SAPO-5, SAPO-8, SAPO-l 1, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, ALPO-5, ALPO-11, ALPO-18, ALPO-31, ALPO-34, ALPO-36, ALPO-37, ALPO-46, which may be metal-containing. Preferred are one or a combination of SAPO-18, SAPO-34, SAPO-35, SAPO-44, SAPO-56, ALPO-18 and ALPO-34, more preferred one or a combination of SAPO-18, SAPO-34, ALPO-34 and ALPO-18, and most preferably one or a combination of SAPO-34 and ALPO-18.

In addition to the possibility of using mixtures of molecular sieves, for example those of different structure types, the molecular sieve may also be an intergrowth material having two or more distinct phases of crystalline structures within one molecular sieve composition. For example, SAPO-18, ALPO-18 and RUW-18 are of AEI framework type, and SAPO-34 is one of a CHA framework type. These may form intergrowths. The molecular sieve may comprise at least one intergrown phase of AEI and CHA framework types; preferably the molecular sieve has a greater amount of CHA framework type than AEI framework type.

The synthesis of molecular sieves for use in the invention is carried out by methods known per se or described in the literature. Generally, synthesis is carried out by hydrothermal crystallization of appropriate sources of the essential framework elements of the molecular sieve, e.g., of aluminum, silicon, phosphorus, as the case may require, if desired or required a template (structure directing agent), and if desired or required a source of non-framework elements.

As an example, the synthesis mixture may be placed in a sealed pressure vessel, optionally lined with an inert plastic such as polytetrafluoroethylene, and heated, under crystallization pressure and temperature, until a crystalline material is formed, and then recovered by filtration, centrifugation and/or decanting.

The molecular sieves, especially the aluminosilicates, are normally used in MTH conversion in their acid form, and if necessary are converted to their acid form either before or after formulation, a procedure carried out in art-recognized ways. For example, treatment with an acid, e.g., HCl, may be employed, or ion-exchange with an ammonium salt, e.g., NH₄NO₃, followed by calcination. However, it has unexpectedly been found that incomplete conversion to the acid form may be advantageous, enhancing catalyst life, as measured by total mass of reactant converted by unit mass of catalyst, although catalyst activity may be slightly reduced. Although not wishing to be bound by any theory, the longer catalyst life may be attributable to a lower rate of coke formation because of a lower density of acid sites.

Among methods of restricting conversion to the acid, or H, form of the molecular sieve, there may be mentioned the use of sub-stoichiometric proportions of acid and, in the case of molecular sieves manufactured with the use of organic molecules as templates (structure directing agents), incomplete ion-exchange with an ammonium salt, followed by calcination.

Incomplete acidification is accordingly advantageously achieved by a process comprising

a) forming a template-containing molecular sieve,

b) mixing the template-containing molecular sieve with a binder and optionally an additional matrix-forming material under conditions resulting in a composition of the desired pore characteristics,

c) ion-exchanging the template-containing molecular sieve, and

d) calcining the template-containing ion-exchanged molecular sieve.

Although in principle mixing with the binder and ion exchange may be carried out in any order, advantageously ion exchange is effected after formulation and preferably after shaping. It is preferable that no calcination under conditions that would decompose template takes place before ion exchange.

As indicated above, the catalyst composition comprises a matrix for the molecular sieve catalyst particles. Numerous procedures for incorporating catalyst particles into a matrix are known in the art, as are materials for forming the matrix. The catalyst particles may be incorporated in the matrix with the latter in solid form or in the form of a suspension, in which case the matrix material is often referred to in the art as a binder.

Materials that may be used, and are often referred to in the art as binders, include various types of hydrated alumina, silica, and/or other inorganic oxides. A suitable alumina-containing suspension is aluminum chlorhydrate. The inorganic oxide suspension acts like glue binding the molecular sieves and other materials such as a solid matrix material, particularly after thermal treatment. Upon heating, the inorganic oxide suspension is converted into an inorganic oxide matrix component.

One or more binders may be combined with other examples of alumina materials, e.g., aluminum oxyhydroxide, boehmite, diaspore, and transitional aluminas, e.g., pseudoboehmite, α-alumina, β-alumina, γ-alumina, δ-alumina, ε-alumina, κ-alumina, and ρ-alumina, aluminum trihydroxides, e.g., gibbsite, bayerite, nordstrandite, doyelite, and mixtures thereof.

The binders may be alumina suspensions, predominantly comprising aluminum oxide, optionally including some silicon. The binders may be peptized alumina made by treating alumina or alumina hydrates, such as pseudoboehmite, with an acid to prepare suspensions or aluminum ion solutions.

Other examples of matrix materials are rare earth metal oxides, other metal oxides including titania, zirconia, magnesia, thoria, beryllia, quartz, silica or suspensions and mixtures thereof, for example silica-magnesia, silica-zirconia, silica-titania, silica-alumina and silica-alumina-thoria. Natural clays, e.g., those from the families of montmorillonite and kaolin may be used. These natural clays include sabbentonites and those kaolins known as, for example, Dixie, McNamee, Georgia and Florida clays. Other examples of matrix materials include: haloysite, kaolinite, dickite, nacrite, or anauxite. The matrix material, especially the clays, may be subjected to modification processes, e.g., calcination and/or acid treatment and/or chemical treatment, before admixture with the molecular sieve catalyst particles.

The catalyst composition may be prepared, as indicated above, by any of the methods described in the art. Advantageously, however, the catalyst particles are combined with the matrix-forming material in a liquid, preferably water, optionally with a plasticizer, to yield a paste. As plasticizer, there may be mentioned one that will be decomposed during any subsequent heat treatment, e.g., calcination. Suitable materials for this purpose include, for example, alkylated cellulose derivatives, hydroxyethylcellulose (HEC), ammonium alginate, polyvinyl pyrrolidone, and polyethylene glycol.

It has been observed that the formulation procedure at this stage, especially in combination with the nature of the matrix-forming material, may determine whether the resulting composition has the features characteristic of catalytic compositions according to the invention.

It is believed that as obtained commercially the Theological properties of apparently identical samples of matrix-forming materials may differ widely. For example, pseudoboehmite, which yields y-alumina on calcination, is obtainable in the form of agglomerates of particles, the speed of deagglomeration when subjected to mechanical breakdown of different samples of which may differ. It may therefore be important to monitor a property of the paste, for example its viscosity, to ensure that peptization of the matrix-forming material has proceeded sufficiently. For acceleration of peptization of certain matrix-forming materials, e.g., alumina, inclusion of an acid, e.g., nitric acid, in a small proportion has been found advantageous. Although the scope of the invention is not to be limited by any theoretical consideration, it is believed that inadequately deagglomerating and breaking down matrix-forming materials may yield a product having a substantial proportion of macropores, thereby adversely affecting the properties of the final catalyst composition. It is readily possible to ascertain by simple routine experiment, for example, mercury porosimetry, whether a particular product has the characteristics of a catalyst composition according to the invention, and hence whether a modification of the process of manufacture, for example extension of the mixing time or intensification of mixing, should be effected.

The uniformly mixed paste may subsequently be shaped, for example by spray drying to yield microspheres, pelletizing or, preferably, by extrusion.

In a preferred procedure for manufacture of the catalyst composition of the invention, an as-synthesized zeolite is converted to the H-form by calcination and ion-exchange. Calcination may take place, for example, at a temperature within the range of 400° C. to 1000° C., advantageously from 500° C. to 700° C., for from 15 minutes to 20 hours, and allowed to cool. The cooled zeolite is then subjected to ion-exchange with an ammonium salt solution, dried, and again calcined. The conversion to the H-form may be carried out before or preferably after formulation and shaping.

The catalyst is advantageously formulated by admixing with the matrix-forming material in the presence of a plasticizer and, optionally, acid, e.g., hydroxyethylcellulose and nitric acid, in, for example, a kneader until the viscosity of the resulting paste remains constant.

The paste is then extruded, for example in a piston extruder, into cylindrical strings, dried, again calcined, and chopped into pieces of a desired length.

Advantageously, the formulated molecular sieve catalyst composition contains from 1% to 99%, preferably from 10% to 90%, more preferably from 10% to 80%, even more preferably from 20% to 70%, and most preferably from 25% to 60% by weight of the molecular sieve based on the total weight of the molecular sieve catalyst composition.

Once the molecular sieve catalyst composition is shaped, and in a substantially dry or dried state, a heat treatment, for example calcination, is advantageously performed to harden and/or activate the composition. Typical temperatures are in the range from 400° C. to 1000° C., preferably from 500° C. to 800° C., and most preferably from 550° C. to 700° C., typically, for from 15 minutes to 20 hours. Calcination may be carried out in, for example, a rotary calciner, fluid bed calciner, or a batch oven.

The compositions of the invention are useful in a variety of processes including: cracking, of for example a naphtha feed to light olefin(s) or higher molecular weight (MW) hydrocarbons to lower MW hydrocarbons; hydrocracking, of for example heavy petroleum and/or cyclic feedstock; isomerization, of for example aromatics such as xylene, polymerization, of for example one or more olefin(s) to produce a polymer product; reforming; hydrogenation; dehydrogenation; dewaxing, of for example hydrocarbons to remove straight chain paraffins; absorption, of for example alkyl aromatic compounds for separating out isomers thereof; alkylation, of for example aromatic hydrocarbons such as benzene and alkyl benzene, optionally with propylene to produce cumeme or with long chain olefins; transalkylation, of for example a combination of aromatic and polyalkylaromatic hydrocarbons; dealkylation; hydrodecylization; disproportionation, of for example toluene to make benzene and para-xylene; oligomerization, of for example straight and branched chain terminal and internal olefin(s); and dehydrocyclization.

Preferred processes are conversion processes including: naphtha to highly aromatic mixtures; light olefin(s) to gasoline, distillates and lubricants; light paraffins to olefins and/or aromatics; and unsaturated hydrocarbons (ethylene and/or acetylene) to aldehydes for conversion into alcohols, acids and esters. The most preferred process of the invention is a process directed to the conversion of a feedstock comprising one or more oxygenates to one or more hydrocarbons, especially olefins.

Preferred oxygenate feedstocks contain one or more alcohol(s), preferably aliphatic alcohol(s) having from 1 to 20 carbon atoms, preferably from 1 to 10 carbon atoms, and more preferably from 1 to 4 carbon atoms. The alcohols may be straight or branched chain alkanols or their unsaturated counterparts.

Examples of oxygenates are methanol, ethanol, n-propanol, isopropanol, methyl ethyl ether, dimethyl ether, diethyl ether, di-isopropyl ether, formaldehyde, dimethyl carbonate, dimethyl ketone, acetic acid, and mixtures thereof. Preferably the feedstock is one or more of methanol, ethanol, dimethyl ether, and diethyl ether, more preferably methanol and dimethyl ether, and most preferably methanol.

The various feedstocks discussed above, particularly a feedstock containing an oxygenate, more particularly a feedstock containing an alcohol, are converted primarily into one or more olefin(s). The olefin(s) or olefin monomer(s) produced from the feedstock typically have from 2 to 30 carbon atoms, preferably 2 to 8 carbon atoms, more preferably 2 to 6 carbon atoms, still more preferably 2 to 4 carbon atoms, and most preferably ethylene and/or propylene. Other examples of olefin monomer(s) are butene-1, pentene-1, 4-methyl-pentene-1, hexene-1, octene-1 and decene-1 and their internally unsaturated isomers as well as the skeletal isomers of those mentioned. Other olefinic monomer(s) include unsaturated monomers, diolefins having 4 to 18 carbon atoms, conjugated or nonconjugated dienes, polyenes, vinyl monomers and cyclic olefins.

In the most preferred embodiment, the feedstock, preferably of one or more oxygenates, is converted in the presence of a composition of the invention into olefin(s) having 2 to 6 carbon atoms, preferably 2 to 4 carbon atoms. Most preferably, olefin(s), alone or combination, are converted from a feedstock containing an oxygenate, preferably an alcohol, most preferably methanol, to the preferred olefin(s) ethylene and/or propylene.

There are many processes used to convert feedstock into hydrocarbons, especially olefins, including various cracking processes such as steam cracking, thermal regenerative cracking, fluidized bed cracking, fluid catalytic cracking, deep catalytic cracking, and visbreaking. The most preferred process is generally referred to as gas-to-olefins (GTO) or alternatively, methanol-to-olefins (MTO). In a GTO process, typically natural gas is converted into a synthesis gas that is converted into an oxygenated feedstock, preferably containing methanol, the oxygenated feedstock being converted in the presence of a molecular sieve catalyst composition into one or more olefin(s), preferably ethylene and/or propylene. In an MTO process, typically an oxygenated feedstock, most preferably a methanol-containing feedstock, is converted in the presence of a molecular sieve catalyst composition of the invention into one or more olefin(s), preferably and predominantly ethylene and/or propylene, often referred to as light olefin(s).

The feedstock may contain one or more diluent(s), for example, helium, argon, nitrogen, carbon monoxide, carbon dioxide, water, essentially non-reactive paraffins (especially alkanes such as methane, ethane, and propane), essentially non-reactive aromatic compounds, and mixtures thereof. The most preferred diluents are water and nitrogen, with water being particularly preferred.

The proportion of diluent in the feedstock may be in the range of from 1 to 99 mole percent, preferably from 1 to 80 mole percent, more preferably from 5 to 50, and most preferably from 5 to 25, based on the total number of moles of from 1 to 99 mole percent.

The process may be carried out in a fixed bed, a fluidized bed (including a turbulent bed), a continuous fluidized bed, a continuous catalyst regeneration, or a continuous high velocity fluidized bed, reactor.

The process may be carried out at a temperature in the range of from 200° C. to 1000° C., advantageously from 250° C. to 800° C., preferably from 250° C. to 750° C., more preferably from 300° C. to 650° C., and most preferably from 300° C. to 450° C.

The process may be carried out at a pressure, based on the partial pressure of the feedstock exclusive of any diluent, in the range of from 0.1 kPaa to 5 MPaa, advantageously from 5 kPaa to 1 MPaa, and most preferably from 20 kPaa to 500 kPaa.

The process may be carried out at a weight hourly space velocity (WHSV), defined as the total weight of the feedstock excluding any diluent per hour per weight of molecular sieve in the molecular sieve catalyst composition in the reaction zone, when a fluidized bed is used, within the range from 1 hr⁻¹ to 5000 hr⁻¹, preferably from 2 hr⁻¹ to 3000 hr⁻¹, more preferably from 5 hr⁻¹ to 1500 hr⁻¹, and most preferably from 10 hr⁻¹ to 1000 hr⁻¹. Preferably, the WHSV is greater than 20 hr⁻¹, and for conversion of a feedstock containing methanol and dimethyl ether is advantageously in the range of from 20 hr⁻¹ to 300 hr⁻¹. The gas velocity is desirably maintained at a level sufficient to keep the catalyst composition in a fluidized state within the reactor. In a fixed bed reactor a lower WHSV is generally used, for example from 0.01 to 100, advantageously 0.05 to 50, preferably 0.1 to 25, hr⁻¹.

Olefins produced by the process of the invention may be used for the manufacture of aldehydes, alcohols, acids and esters, especially vinyl acetate, higher linear alpha olefins, ethylene dichloride, vinyl chloride, ethylbenzene, ethylene oxide, cumene, isopropyl alcohol, acrolein, allyl chloride, propylene oxide, acrylic acid, ethylene-propylene rubbers, and acrylonitrile. They may also be polymerized to polyolefins and other polymers.

### EXAMPLES

The following examples illustrate the invention. All parts and percentages are by weight unless stated otherwise.

### Example 1

A catalyst composition was formed by adding to 33.5 parts of an 8.5% aqueous solution of hydroxyethylcellulose 15 parts of as-synthesized ZSM-5 having a Si/Al atomic ratio of 39, and 30 parts of pseudoboehmite (Pural SB1) in a kneader (Haake, Polydrive). After 30 minutes of kneading at 40 r.p.m. the average torque remained constant at 10 to 15 Nm and the resulting paste was recovered.

The paste was transferred to a piston extruder, shaped to cylindrical strings of 2 mm diameter, and left to dry overnight at ambient temperature. The dried extrudate was calcined in a ventilated oven by heating at 3° C. per minute to 550° C., maintained at that temperature for 4 hours, and allowed to cool to room temperature. The cooled extrudate strings were then chopped into 5 mm lengths.

By this calcination, all template remaining from the zeolite synthesis was removed, and the pseudoboehmite binder converted to a γ-alumina matrix. The catalyst was then ion-exchanged with an ammonium chloride solution, and the formulated catalyst recovered by filtration. After repeating the exchange procedure twice, the formulated catalyst was re-suspended in distilled water until the liquid was free from chloride ions, and dried overnight at 120° C. Finally, the calcination process was repeated to yield the H-form of the zeolite catalyst.

The above procedures were repeated using ZSM-5 of the same Si:Al ratio but of particle sizes 800 nm and 650 nm. The crush strengths of the samples were measured according to ASTM D4179. Porosity and mean pore size were determined by mercury porosimetry using a Micromeretics Autopore III. The results are summarized in Table 1 below.

TABLE 1

| | Zeolite | | | Mean Pore |
|---|---|---|---|---|
| | Particle Size, | Crush | Porosity | Diameter |
| Sample | nm | Strength N | % | nm |
| 1 | 3500 | 38 | 59 | 12 |
| 2 | 800 | 42 | 56 | 11 |
| 3 | 650 | 41 | 57 | 11 |

The distribution of mesopores was monomodal in all cases, 100% of the pore volume being in mesopores smaller than 15 nm.

### Example 2 and Comparative Example A

It is known that certain prior art molecular sieve catalyst compositions, especially commercial extrudates in which the matrix is alumina, exhibit at least bimodal pore distribution, probably because they are formulated by processes of limited efficiency in breaking down agglomerates of alumina particles. For example, it has been observed that one commercially available pseudoboehmite is in the form of agglomerates of diameter in the range of 25 μm to 250 μm; these are apparently obtained by spray-drying of polycrystalline primary particles of diameters about 45 nm; these in turn contain primary crystals of about 5 nm. It is believed that incomplete breaking down of the agglomerates leads to a substantial proportion of pore volume being in the form of macropores, and also mesopores being of two different median pore sizes. For comparison, therefore, with the catalyst compositions of the invention, an extrudate was formulated to replicate a commercial material. A catalyst composition was formed by adding to 37 parts of an 8.5% aqueous solution of HEC 18.5 parts of as-synthesized ZSM-5, silicon:aluminum ratio 32:1, particle size 300 to 500 nm, and 37 parts of pseudoboehmite, Pural SB). The samples were kneaded for 15 minutes (comparison A) and 45 minutes (Example 2) and extruded and calcined as described in Example 1. The results are shown in Table 2.

TABLE 2

| | Kneading Time, | Porosity | Crush | Mean Pore |
|---|---|---|---|---|
| Example | M in | % | Strength, N | Diameter nm |
| Comp. A | 15 | 54.1 | 46 | 6/10/100/600 |
| 2 | 45 | 53.5 | 59 | 10 |

The product of Comparison Example A had four peaks in the plot of pore size, as measured by mercury porosimetry, with 30% of the pore volume represented by macropores of 100 and 600 nm diameter, although the total porosity was very similar to that of the product of Example 2. The comparison Example also had a substantially lower crush strength.

### Example 3

Products formulated in the way described in Example 2 and Comparison Example A were used to catalyse the methanol to olefins process.

5 parts of each catalyst composition were mixed with a similar volume of SiC particles of diameter 0.2 mm and charged to a tubular reactor. Upstream and downstream of the catalyst bed the reactor was loaded with 0.5 mm silicon carbide particles. The high thermal conductivity of SiC reduces any axial temperature gradient in the catalyst bed and the upstream loading ensures a plug flow profile and a desired feed gas temperature on contacting the catalyst bed.

The composition of the product stream was analysed, after injection of neopentane as reference gas stream, by a gas chromatograph (Hewlett Packard GC 6890 with Cp-Porabond Chrompack 7354 column and FID detector).

After heating the catalyst bed to 360° C. while purging with an N₂ stream, the process was carried out at temperatures ranging from 300 to 420° C., WHSV 1 to 7 h⁻¹ (based on methanol to total catalyst composition) an inlet pressure of 1.65 bar, methanol partial pressure 0.354 bar, carrier gas nitrogen, and co-feed internal standard 0.011 mmol/min.

The performance characteristics, conversion, selectivity, and yield are calculated on a carbon basis. Since the only reactive component in the feedstream is methanol (which may be converted under the reaction conditions to dimethyl ether), the yield of a product species is directly related to its concentration in the product stream.

Table 3 shows the results at 320° C. at various WHSV's.

TABLE 3

| | Product without | Product with |
|---|---|---|
| WHSV (h⁻¹) | macropores | macropores |
| 2.5 | 0.73 | 0.63 |
| 3.5 | 0.58 | 0.43 |
| 4.5 | 0.47 | 0.33 |
| 5.5 | 0.39 | 0.25 |
| 6.3 | 0.33 | 0.20 |

The results show that, at a given WHSV, the catalyst of the invention gives a conversion substantially higher than that of the replicated commercially made catalyst. Alternatively, at a given conversion rate, a substantially higher space velocity may be employed. Selectivities to ethylene were similar. Selectivities to ethylene and propene using the product according to the invention at conversions of 31% and 24.3% were 48.2% and 54%, while using the comparison sample at conversions of 32.9% and 25.2% gave selectivities to ethylene and propylene of 50.1% and 55.2%.

### Example 4

In this example, the catalyst lifetimes of two compositions according to the invention were compared; the first having been ion-exchanged after calcination, the second having been ion-exchanged with template retained, so that the concentration of acid sites in the second was less than that of the first, the two compositions being otherwise identical, being extrudates of 61% γ-alumina and 39% ZSM-5. The two catalyst compositions were used in the methanol to olefin conversion process at 320°, the first sample being used at a WHSV of 0.5 h⁻¹ and the second at the lower WHSV of 0.1 h⁻¹, to compensate for the lower acid site concentration.

The first composition began to deactivate after about 80 hours, and after about 270 hours no conversion of methanol was observed. In contrast, the second composition had not deactivated after 1200 hours, when the trial was stopped. In terms of methanol conversion, the first composition started to deactivate after 40 kg of methanol per kg of catalyst had been treated, and deactivation was complete after contact with 270 kg. The second, partly exchanged, composition had converted 120 kg per kg catalyst with no indication of any deactivation.

### Examples 5 to 8

A catalyst composition was formed by adding to 33.5 parts of HEC (8.5% aqueous solution) 15 parts of H-ZSM-5 and 30 parts of pseudoboehmite (Pural SB 1) in a kneader. Different portions of the composition were kneaded for different times before extruding as described in Example 1. The results were as follows:

TABLE 4

| | Mixing Time, | | Mean Pore |
|---|---|---|---|
| Example | Mins | Porosity % | Diameter |
| 5 | 15 | 55.6 | 10.0 |
| 6 | 30 | 55.9 | 10.3 |
| 7 | 45 | 58.3 | 10.6 |
| 8 | 60 | 58.1 | 10.6 |

The distribution of mesopores was monomodal in all cases. The difference between Comparison A and Example 5 is striking. It is believed that it is due to the difference in characteristics between Pural SB and Pural SB1, the latter having been manufactured in a manner that allows its rapid peptization in water.

### Example 9

A catalyst composition was formed by adding to 33.5 parts of HEC (8.5% aqueous solution) 15 parts of H-ZSM-5 having a Si/Al atomic ratio of 32 and 30 parts of pseudoboehmite (Pural SB1) in a kneader. The composition was kneaded for 30 minutes before extruding as in example 1.

The catalyst was then used in two experiments using methanol flow interruptions during the catalytic conversion of methanol to olefins in the reactor described in example 3.

The reaction unit used consists of a fixed bed reactor, on-line GC for product analysis, and a catalytic afterburner downflow. The analyses of the CO₂ and CO concentrations in the off-gas of the afterburner allowed for a continuous monitoring of the carbon balance. Experiments were carried out with a N₂/methanol feed at a fixed temperature of 320° C., a total pressure of 1.65 bar, and a methanol partial pressure of 0.354 bar. By the use of a by-pass it was possible to periodically channel the feed flow directly towards the analytical part (GC and afterburner) of the unit without contact to the reactor. During by-pass of the reactor, the catalysts remained within a stagnant mixture of both, unreacted feed and former reaction products.

Transport properties of the catalyst were altered by variation of the pore size distribution of the gamma-Al₂O₃ matrix, and by the size of the particles. The effect of external mass transport was studied by variation of the flow velocity at constant residence time (as related to the catalyst mass). Dilution of the catalyst with SiC particles of 0.2 mm diameter in a ratio 1:1, and the complete filling of the reactor volume (L=350 mm, Ø=15 mm) with SiC particles up- and downflow to the catalyst bed ensured plug flow and efficient heat transfer.

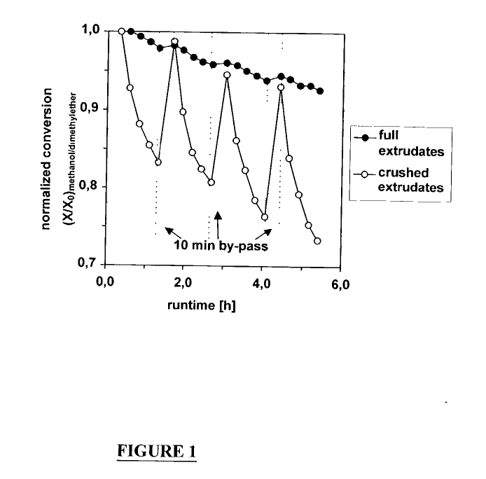

FIG. 1 shows results of two experiments with periodical flow interruptions of 10 minutes after 15 min time on stream. Because the gamma-Al₂O₃ matrix is catalytically active in the conversion of methanol to dimethylether (DME), we consider these compounds together as one pseudo-reactant in the calculation of the conversion. Residence time and catalyst were the same in both runs. However, complete extrudates were used in one experiment (filled circles), whereas the extrudates were crushed down to small particles (350-500 μm) for the other (hollow circles). The data show that the catalysts deactivate during time on stream, and that they recover while they are resting in a stagnant reaction mixture (by-pass). It can clearly be observed that this behavior and the corresponding sawtooth profile are more pronounced when the smaller particles are used.

Obviously, the deactivation cannot be explained by coking alone. Coking is irreversible and accounts only for the over-all decrease in activity as indicated by the falling trend of the sawtooth profile. The sawteeth themselves, however, represent a reversible deactivation on stream, and a “self-regeneration” at stagnant conditions. Many authors before postulated the existence of an autocatalytic intermediate in the MTO reaction. We suppose that the depletion of such an autocatalytic species is responsible for the reversible activity drop. During by-pass of the reactor, this species can accumulate again because transport into the bulk fluid is slowed-down. Our assumption was confirmed by experiments with different flow velocities (check on external mass transfer effects), and with catalysts of equal size, exhibiting different pore size distributions in the matrix (check on internal mass transfer effects). The results showed that high flow rates as well as large pores promote a pronounced (reversible) deactivation.

This is an exceptional example of mass transport limitations having a beneficial rather than a negative effect on catalyst performance.

By means of reactor operation with periodical flow interruption we have shown that the concentration of an autocatalytic intermediate within the catalyst is decisive for its activity. The rate of the transport through the pores and subsequently into the bulk fluid must match with the rate of formation in order to prevent depletion of the autocatalytic species and the concomitant drop in activity.

Having now fully described this invention, it will be appreciated by those skilled in the art that the invention can be performed within a wide range of parameters within what is claimed, without departing from the spirit and scope of the invention.

1. A catalyst composition comprising molecular sieve particles in a matrix, at least 75% of the pore volume of the composition, as measured by mercury porosimetry, having a pore size of at most 20 nm.

2. A composition as claimed in claim 1, wherein at least 90% of the pore volume has a pore size of at most 20 nm.

3. A composition as claimed in claim 1, wherein the mean pore size is within the range of from 4 to 15 nm.

4. A composition as claimed in claim 1, wherein the pore size distribution is monomodal.

5. A composition as claimed in claim 1, wherein the molecular sieve particles consist of crystalline silicoaluminate molecular sieve.

6. A composition as claimed in claim 5, wherein the crystalline silicoaluminate molecular sieve is of one or more of a group of framework types consisting of AEL, AFI, BEA, CHA, EDI, FAU, FER, GIS, LTA, LTL, MER, MFI, MOR, MTT, MWW, TAM and TON.

7. A composition as claimed in claim 5, wherein the crystalline silicoaluminate molecular sieve is ZSM-5.

8. A composition as claimed in claim 5, wherein the crystalline silicoaluminate molecular sieve is completely in an acid form.

9. A composition as claimed in claim 5, wherein the crystalline silicoaluminate molecular sieve is only partially in an acid form.

10. A composition as claimed in claim 1, wherein the molecular sieve particles consist of an aluminophosphate or silicoaluminophosphate molecular sieve.

11. A composition as claimed in claim 10, wherein the aluminophosphate or silicoaluminophosphate molecular sieve is selected from one or more of the group consisting of SAPO-18, SAPO-34, SAPO-35, SAPO-44, SAPO-56, ALPO-18 and ALPO-34.

12. A composition as claimed in claim 10, wherein the aluminophosphate or silicoaluminophosphate molecular sieve is an intergrowth or mixed phase of AEI and CHA framework type molecular sieves.

13. A catalyst composition as claimed in claim 1, wherein the composition comprises alumina.

13. A catalyst composition as claimed in claim 1, in the form of an extrudate.

14. A catalyst composition as claimed in claim 1, in the form of fluidizable catalyst particles.

15. A process for the manufacture of the catalyst composition as claimed in claim 1, which comprises mixing molecular sieve particles with a binder and optionally an additional matrix-forming material under mixing conditions such that at least 75% of the pore volume, as measured by mercury porosimetry, of the composition has a pore size of at most 20 nm.

16. A process for the manufacture of a catalyst composition comprising molecular sieve catalyst particles in a matrix, at least 75% of the pore volume of the composition, as measured by mercury porosimetry, being of pore size of at most 20 nm, the process comprising the steps of:
a) forming a template-containing molecular sieve,
b) mixing the template-containing molecular sieve with a binder, and optionally an additional matrix-forming material, under conditions resulting in the catalyst composition of the desired pore volume and pore size,
c) ion-exchanging the template-containing molecular sieve, and
d) calcining the template-containing ion-exchanged molecular sieve.

17. A process as claimed in claim 16, wherein step c) is performed after step b).

18. A process for the conversion of a feedstock to a hydrocarbon-containing product, the process comprising contacting the feedstock with a catalyst composition of claim 1.

19. A process as claimed in claim 18, wherein the feedstock is an oxygenate-containing feedstock.

20. A process as claimed in claim 18, wherein the hydrocarbon-containing product is an olefin-containing product.

21. A process as claimed in claim 20, wherein the feedstock is methanol and/or dimethylether and the hydrocarbon-containing product is ethylene and/or propylene.

22. A process for enhancing the conversion rate in converting a feedstock to a hydrocarbon-containing product using the catalyst composition of claim 1.

23. A process for enhancing catalyst life in an oxygenate to hydrocarbon conversion process using a catalyst composition of claim 1 that is only partially in acid form.

24. A process to reduce deactivation rate in an oxygenate to hydrocarbon conversion process using a catalyst composition of claim 1 that is only partially in acid form.
